(19) United States
(12) Patent Application Publication
(10) Pub. No.: US 2012/0172821 A1
(43) Pub. Date: Jul. 5, 2012

(54) Therapeutic clothing and related methods

(76) Inventors: Jeffrey A. McKinney, Lafayette, CA (US); Susan L. Wooldridge, Lafayette, CA (US)

(21) Appl. No.: 13/374,552

(22) Filed: Jan. 3, 2012

Related U.S. Application Data
(60) Provisional application No. 61/460,457, filed on Jan. 3, 2011.
(60) Provisional application No. 61/464,726, filed on Mar. 8, 2011.

(51) Int. Cl. A61M 35/00; B32B 3/10; A43B 17/00; A41D 23/00; A41D 10/00; A41B 9/06; A42B 1/00; B32B 5/02; A41D 19/00

(52) U.S. Cl. 604/290; 428/196; 2/239; 2/159; 2/69; 2/113; 2/171; 2/207

(57) ABSTRACT

The present invention is related to articles of clothing that can be used to lessen the symptoms of particular diseases and/or skin conditions, designs of the clothing, and methods and tools for commercializing and marketing the clothing. In one aspect, the present invention is directed to an article of clothing. The article of clothing includes at least a first coating. The first coating decreases the wicking or absorbance of water as compared to clothing that does not include the coating.

This application claims priority under 35 U.S.C. §119(e) to U.S. provisional patent application Ser. No. 61/460,457, filed Jan. 3, 2011, and to U.S. provisional patent application Ser. No. 61/464,726, filed Mar. 8, 2011, both of which are hereby incorporated by reference.

### FIELD OF THE INVENTION

The present invention is related to articles of clothing that can be used to lessen the symptoms of particular diseases and/or skin conditions, designs of the clothing, and methods and tools for commercializing and marketing the clothing.

### BACKGROUND OF THE INVENTION

There have been several reports directed to medically-related clothing.

U.S. Pat. No. 4,882,392, for instance, discusses radiopaque materials comprising heavy metal salts, such as bismuth and uranium salts complexed with a polymer. The metallic complexes, which are permanent, nonleachable, and have radiopacities at least equivalent to that of aluminum, do not adversely affect the mechanical and physical properties of compositions. They are thought to be useful as medical and dental resins, in fabricating medical and dental appliances, prosthetic devices, radiation shielding devices and radiopaque polyester fabrics for clothing.

U.S. Pat. No. 5,652,049 reports composite non-woven fabrics. The fabrics comprise at least a first ply that is an antibacterial fibrous web, and a second ply comprising a web of non-woven non-antibacterial fibers serving as a “base structure” providing strength and dimensional stability to the fabric. Each ply adheres strongly to adjacent plies of the fabric by inter-entanglement of the fibers in the respective layers with each other, which defines an intensity gradient of antibacterial property across the thickness dimension of the fabric. The fabrics can be made sufficiently soft and mild for skin contact, and have many potential uses such as in clothing, medical fabrics, sheets, and the like. The fabrics have the same or a lesser amount of antibacterial fibers than prior-art non-woven antibacterial fabrics without adversely affecting strength, dimensional stability, or antibacterial efficacy.

U.S. Pat. No. 6,460,187 presents multipurpose medical clothing, such as a gown or robe, that safely accommodates medical appliances attached to a patient, affords privacy, and therefore encourages the patient to be up and ambulatory. An illustrated gown embodiment includes rectangular front and back panels open at the sides, belting, inside and outside pockets, and inside and outside hangers. The panels are of equal width greater than average human shoulder width to provide sleeves when worn. The belting extends from the back panel around the sides and is tied in front. The front panel is slightly shorter than the back panel so that the panels are of about the same height when the gown is worn and belted. The pockets are attached to the inside and outside of the front panel below the belt line at substantially the same height and in transversely spaced relation to each other. The hangers are attached to the inside and outside of the front panel respectively below the inside and outside pockets. The gown accommodates various medical appliances such as: a telemetry transmitter with its sensors attached to patient and its leads passing under the tied belting to relieve the pressure on the sensors; a catheter unit hung from either the inside or outside hangers, and thus below the patient's bladder, with its tubing coiled and supported on the hangers; and IV tubing threaded through one of the loops formed by a hangers and thus supported between an adjacent mobile stand and the patient.

Despite the above reports, there remains a need in the art for new types of medically-related clothing.

### SUMMARY OF THE INVENTION

The present invention is related to articles of clothing that can be used to lessen the symptoms of particular diseases and/or skin conditions, designs of the clothing, and methods and tools for commercializing and marketing the clothing.

In one aspect, the present invention is directed to an article of clothing. The article of clothing includes at least a first coating. The first coating decreases the wicking or absorbance of water as compared to clothing that does not include the coating.

In another aspect, the present invention is directed to an article of clothing. The article of clothing includes a first section; the first section includes a first coating. The first coating decreases the wicking or absorbance of water as compared to a second section that does not include the first coating.

In another aspect, the present invention is directed to a method of treating skin dryness. The method involves wearing an article of clothing according to the present invention.

In another aspect, the present invention is directed to marketing an article of clothing according to the present invention. The method involves: a) taking fabric from an article of clothing according to the present invention, where the clothing has a section that will absorb water and a section that absorbs water much less readily; b) sprinkling water, either containing a dye or not, over the fabric; c) allowing prospective customers to readily observe that one section of the fabric absorbs water readily while the other section does not.

### BRIEF DESCRIPTION OF THE FIGURES

FIG. 1 shows a coating pattern for an article of clothing of the present invention where the pattern involves solid coating sections.

FIG. 2 shows another coating pattern for an article of clothing of the present invention, where the pattern involves striped coating sections.

FIG. 3 shows another coating pattern for an article of clothing of the present invention where the pattern involves checked coating sections.

FIG. 4 shows another coating pattern for an article of clothing of the present invention where the pattern involves triangular coating sections.

FIG. 5 shows another coating pattern for an article of clothing of the present invention where there are two different coatings, one that decreases water-wicking ability and another that increases water-wicking ability.

FIG. 6 shows a design for an article of clothing according to the present invention, where the design reflects the coating pattern of the clothing.

### DETAILED DESCRIPTION

The present invention is related to articles of clothing that can be used to lessen the symptoms of particular diseases and/or skin conditions, designs of the clothing, and methods and tools for commercializing and marketing the clothing.

The articles of clothing that can be used to lessen the symptoms of particular diseases and/or skin conditions according to the present invention include, without limitation, the following: socks, underwear, gloves, hats, sleep wear, pants, shirts, under shirts, camisoles, tee shirts, and scarves.

The articles of clothing include a coating that serves to decrease absorbance or the water wicking properties of the clothing as compared to clothing that does not include the coating. The coating is preferably on the side of the clothing proximal to the wearer's skin.

The coating is of any suitable composition that serves to decrease the water-wicking properties of a fabric, although the composition oftentimes includes fluorocarbon-based polymers. Nonlimiting examples of such coatings/compositions include: Scotchgard; Teflon; Resists Spills; the coatings discussed in Appl. No. PCT/US1999/020926; the coatings discussed in the Journal of Industrial Textiles, Vol. 32, No. 2, 119-138 (2002); the coatings discussed in U.S. Pat. No. 5,747,392; silicone elastomers produced by Dow Corning and other companies; coatings discussed in U.S. Pat. No. 6,372,674; coatings discussed in U.S. Pat. No. 5,874,148; coatings discussed in U.S. Pat. No. 6,165,920; Nikiwax; ReviveX; Tectron; Kiwi Camp Dry; coatings discussed in U.S. Pat. Nos. 5,565,265, 6,024,823, 7,531,219, and WO/2005/103362; and Granger's; coatings discussed in U.S. Pat. No. 6,372,674. The preceding documents in this paragraph are incorporated-by-reference for all purposes into this application. In certain cases, the composition includes a fluorocarbon-based polymer and the polymer is a polytetrafluoethylene polymer such as ZONYL® PTFE.

Nonlimiting examples of fabrics including a coating are: eVent fabrics; Nikiwax Directional Textiles as used by Paramo Directional Clothing Systems; Fur Technology supplied by FurTech; Gore-Tex; Sympatex; 3-Ply Entrant-EV membrane Mountain Equipment Co-op; AquaNix by Taiga Works; Entrant by Toray Industries; Drilite Extreme, made by Mountain Equipment; HyVent, made for the North Face by Toray; H2No made for Patagonia, Inc. by Toray; Omni-Tech by Columbia Sportswear; Conduit made by Mountain Hardwear; Triplepoint Ceramic made by Lowe Alpine; Precip, made by Marmot; Watergate made by Outdoor Designs; Closely woven cotton; Polymer-encapsulated cotton; EPIC made by Nextec; Wax impregnated cotton or cotton/polyester mixes (such as those used in Barbour coats); Epoxy Hyper-Dri by Mark's Work Wearhouse; Ventile by Ventile; Trilogy by Ripzone; Stormtech by Peter Storm; and, c change by Schoeller textiles.

The “hand feel” of the coated fabric, or coated portion of the fabric, is not substantially different from that of the uncoated fabric. Hand feel relates to the smoothness or softness of fabric, and it is typically measured using a standardized American Association of Textile Chemists and Colorists (i.e., AATCC) method: AATCC Method EP-5, “Fabric Hand: Guidelines for the Subjective Evaluation of”. Typically, the hand feel of the coated fabric or portion measured by AATCC EP-5 is less than 50.0 percent different from that of uncoated fabric. In certain cases, the hand feel is less than 45.0 percent, 40.0 percent, 35.0 percent, 30.0 percent different from the uncoated fabric. In other cases it is less than 25.0 percent, 20.0 percent, 17.5 percent, 15.0 percent, 12.5 percent, 10.0 percent, 7.5 percent or 5.0 percent different.

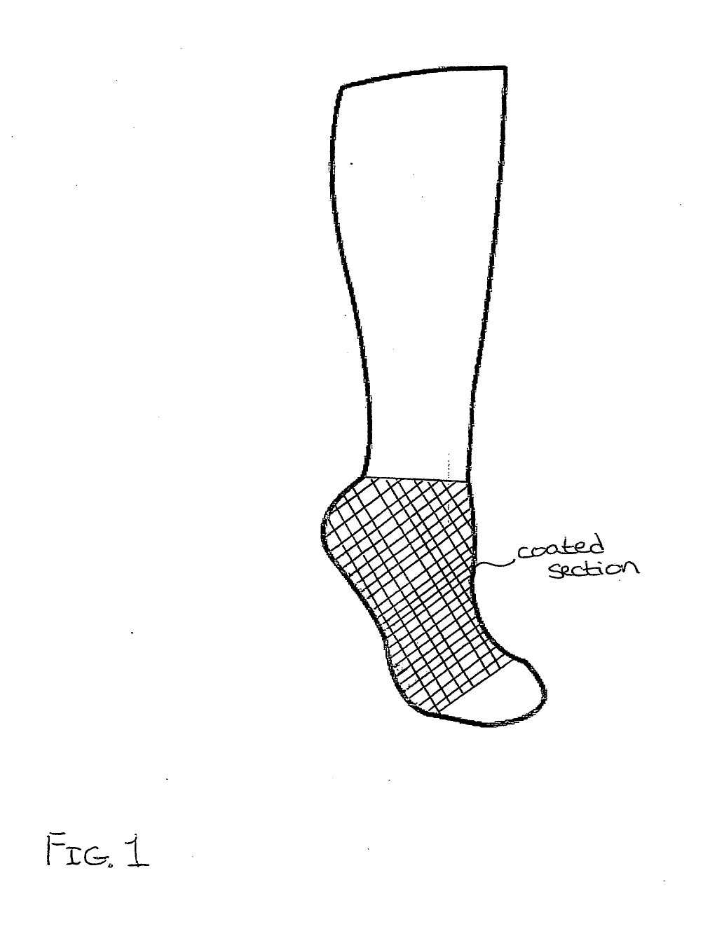

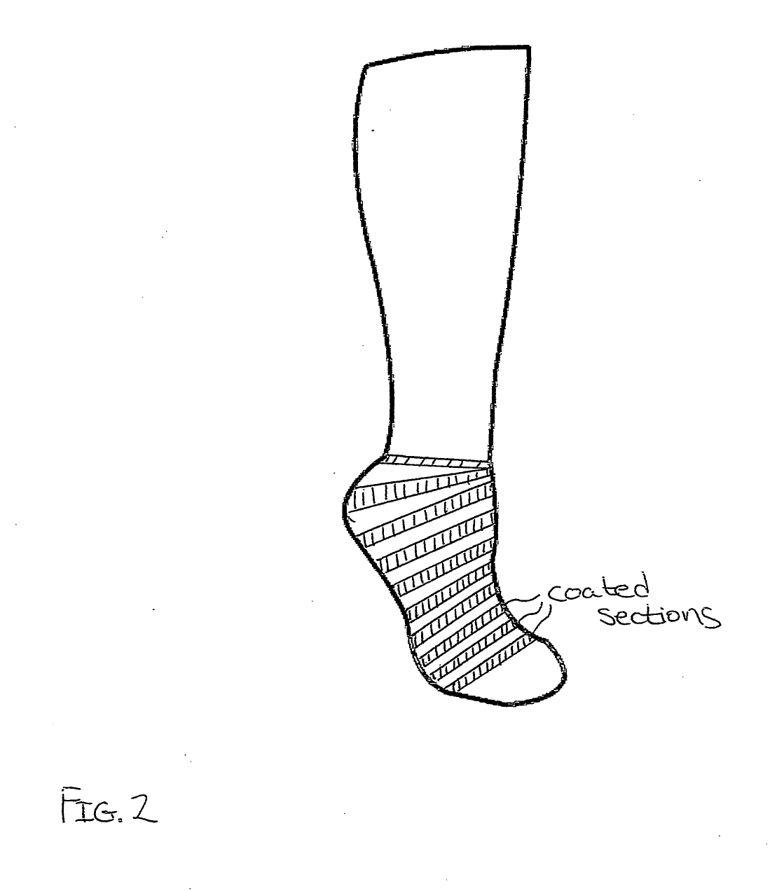

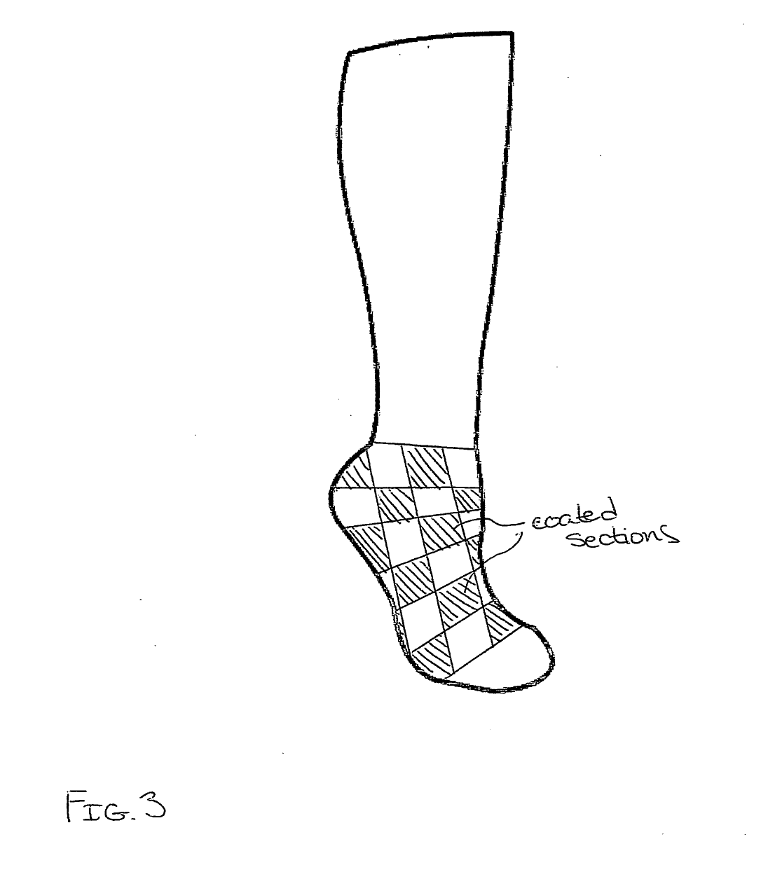

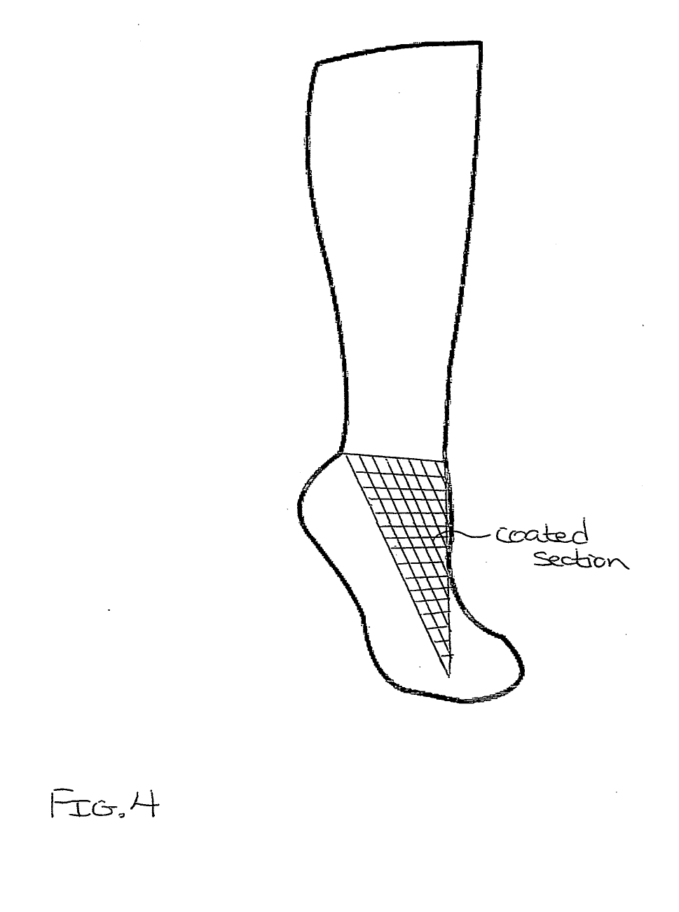

The fabric making up the article of clothing is typically coated in a pattern rather than wholly coated with a coating that decreases the water-wicking ability of the fabric (although some clothing articles are wholly coated). FIG. 1, for instance, shows a sock that includes a coating in sections, specifically in a section beginning behind the toes and ending just below the ankle. FIG. 2 shows a sock that includes the coating as striped sections. FIG. 3 shows a sock that includes the coating as checked sections; and, FIG. 4 shows a sock that includes the coating in one or more triangular sections.

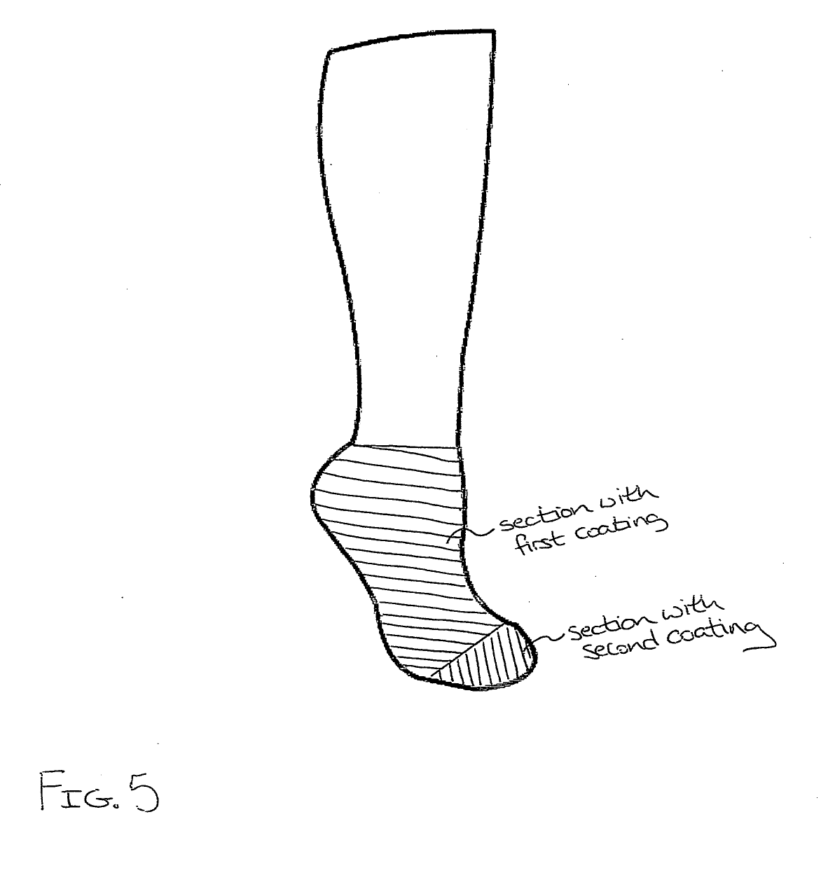

The article of clothing may furthermore include the coating decreasing water-wicking ability and a second coating. For example, the second coating can be a different coating decreasing water-wicking ability. In another example, the second coating can be a coating that increases water-wicking ability. In another example, the second coating can be an antimicrobial coating or an antimicrobial coating that also increases water-wicking ability. FIG. 5 shows a sock that includes two coatings—one that decreases water-wicking ability and another that increases water-wicking ability.

Non-limiting examples of antimicrobial coatings include: coatings containing silver salts; coatings containing cetylpyridinium chloride; benzyldimethylhexadecyl ammonium chloride; and, BioSmartâ from Milliken.

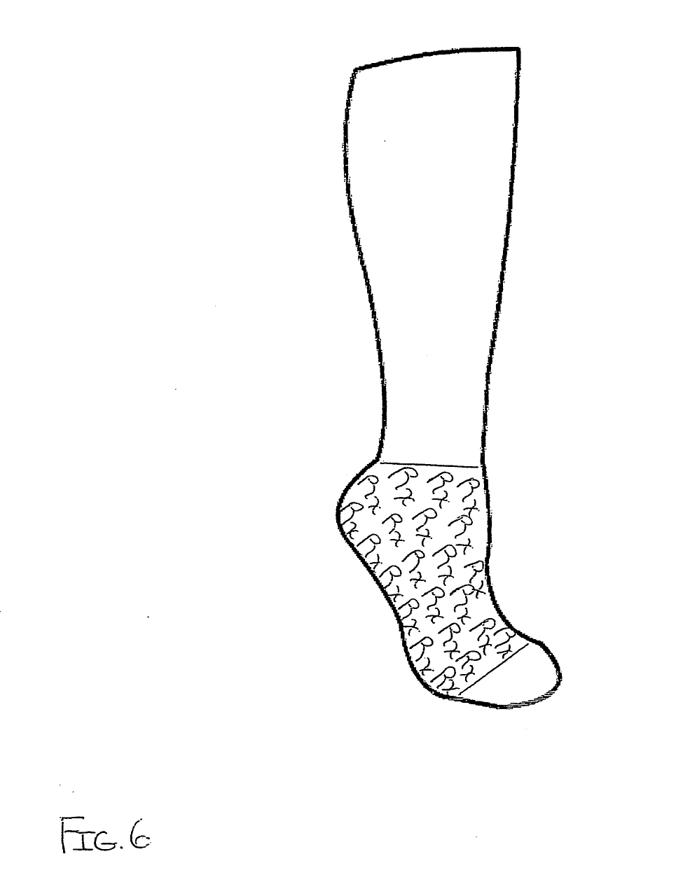

Articles of clothing of the present invention may furthermore include designs, preferably designs that are reflective of the coating placement. One such design is shown in FIG. 6, where the coated portion of the sock includes the repeated term “Rx.”

The articles of clothing can be sold or marketed in a variety of ways. For example, the clothing can be sold at a retail store, in a doctor's office, or through the internet. Where the sale occurs through an in-person transaction (e.g., retail store or doctor's office), the sale can be facilitated through a demonstration of the clothing's functional qualities. An example of such a demonstration is: a) taking fabric from an article of clothing as shown in FIG. 1, where the clothing has a section that will absorb water and a section that absorbs water much less readily; b) water, either containing a dye or not, is sprinkled over the fabric; c) prospective customers can readily observe that one section of the fabric absorbs water readily while the other section does not. Where the demonstration does not occur through an in-person transaction (e.g., internet), the same type of demonstration can be shown through video or a series of photographs.

Diseases where symptoms can be lessened by using articles of clothing according to the present invention include, without limitation, the following: diabetes, which can produce dryness in the extremities, especially the feet and hands; psoriasis; and eczema. The clothing is also used to lessen skin dryness.

Skin dryness is measured on a 5 point scale: 0=smooth, no evidence of dryness; 1=slightly dry skin, occasional scale, not necessarily uniformly distributed; 2=moderately dry skin, uniformly distributed scale, but no widespread uplifting flaking; 3=severely dry skin, pronounced scaling visible with the naked eye, definite uplifting of edges or scale sections—skin surface may have a whitish appearance; 4=extremely dry skin, more scale and pronounced separation of scale edges,some evidence of cracking (for hands, the skin looks abraded). Values are typically assigned by a dermatologist or other physician, but they may also be made by a one or more non-medical personnel. A determination of a group skin dryness value is made based on the evaluation of the same body part in at least 5 individuals.

Where an individual is experiencing dry skin, wearing clothing that includes at least a coated portion that touches the area of dryness will improve one's skin dryness score. After wearing such clothing for 7 days, the dryness score will typically improve by at least 5%. In certain cases, the score will improve by at least 7.5%, 10.0%, 12.5%, 15.0%, 17.5%, 20.0%, 22.5% or 25%. In other cases, the score will improve by at least 30.0%, 35.0%, 40.0%, 45.0% or 50.0%.

After wearing such clothing for 14 days, the dryness will typically improve by at least 10%. In certain cases, the score will improve by at least 15.0%, 20,0%, 25%, 30.0%, 35.0% or 40.0%. In other cases, the score will improve by at least 45.0%, 50.0%, 55.0%, 60.0%, 65.0% or 70.0%.

### EXAMPLE

A 48 year old male subject was experiencing severely dry skin on the sides and bottoms of both feet. Socks completely coated with a fluorocarbon-based coating according to the present invention were worn by the subject for 7 days. On the 8^{th} day, the subject's feet were evaluated. Dryness of both feet was evaluated to be moderately dry in contrast to the starting state of severely dry.

1. An article of clothing, wherein the article of clothing comprises a first section, and wherein the first section comprises a first coating, and wherein the first coating decreases the wicking or absorbance of water as compared to a second section that does not comprise the first coating.

2. The article of clothing according to claim 1, wherein the first coating is in a striped pattern in the first section.

3. The article of clothing according to claim 1, wherein the first coating is in a checked pattern in the first section.

4. The article of clothing according to claim 1, wherein the first coating is in a triangular pattern in the first section.

5. The article of clothing according to claim 1, wherein the article of clothing is a sock.

6. The article of clothing according to claim 1, wherein the article of clothing is a glove.

7. The article of clothing according to claim 1, wherein the article of clothing is sleepwear.

8. The article of clothing according to claim 1, wherein the article of clothing is an undershirt.

9. The article of clothing according to claim 1, wherein the article of clothing is a hat.

10. The article of clothing according to claim 1, wherein the article of clothing is a scarf.

11. The article of clothing according to claim 1, wherein the second section comprises a second coating.

12. The article of clothing according to claim 1, wherein the second section comprises a second coating, and wherein the second coating is an antimicrobial coating.

13. The article of clothing according to claim 1, wherein the second section comprises a second coating, and wherein the second coating increases water wicking or absorbency relative to the clothing where no coating is included.

14. The article of clothing according to claim 1, wherein the second section comprises a second coating, and wherein the second coating both increases water wicking or absorbency relative to the clothing where no coating is included and is antimicrobial.

15. A method of treating skin dryness, wherein the method comprises wearing the article of clothing of claim 1.
